(19) United States
(12) Patent Application Publication
(10) Pub. No.: US 2025/0180534 A1
(43) Pub. Date: Jun. 5, 2025

(54) Food condition tracking method based on spectral images, food management method, and food management device supporting same

(71) Applicant: SK Planet Co., Ltd., Seongnam-si (KR)

(72) Inventor: Yeonghyeon PARK, Suwon-si (KR)

(21) Appl. No.: 19/047,631

(22) Filed: Feb. 7, 2025

Related U.S. Application Data
(63) Continuation of application No. PCT/KR2023/019419, filed on Nov. 29, 2023.

(30) Foreign Application Priority Data
Dec. 6, 2022 (KR) 10-2022-0168777
Dec. 6, 2022 (KR) 10-2022-0169198

(51) Int. Cl. G01N 33/02; G01N 21/17; G01N 21/27; G01N 21/84; G06Q 10/0875; G06Q 50/02

(52) U.S. Cl. CPC G01N 33/025; G01N 21/27; G06Q 10/0875; G06Q 50/02; G01N 2021/1776; G01N 2021/8466

(57) ABSTRACT

The present invention relates to a food condition tracking method based on spectral images, a food management method, and a food management device supporting same. The present invention may provide a food condition tracking method based on spectral images, the method comprising the steps in which a processor of a food management device: forms a communication channel with a storage warehouse in which the food is stored; receives, from the storage warehouse, food-related information about food stocked in the storage warehouse; acquires spectral images of the food at regular intervals using a spectroscopic camera arranged so as to able to capture spectral images of the food; and monitors changes in the condition of the food by analyzing the obtained spectral images. The present invention may also provide a food management device supporting the method.

### CROSS-REFERENCE TO RELATED APPLICATIONS

This is a bypass continuation of International PCT Application No. PCT/KR2023/019419, filed on Nov. 29, 2023, which claims priority to Korean Patent Application No. 10-2022-0168777, filed on Dec. 6, 2022, and Korean Patent Application No. 10-2022-0169198, filed on Dec. 6, 2022, which are incorporated by reference herein in their entirety.

### TECHNICAL FIELD

The present invention relates to food state tracking and management, and more particularly, to a technology that supports more accurate tracking of changes in food or adaptive processing of management of ripened food using spectral images.

### BACKGROUND ART

Food after produced and processed can be stored until it reaches the consumer's table. In order to prevent food from being deformed or spoiled, warehouses for storing food are designed to maintain temperatures at a certain level or less so that items such as food and beverages can be stored for a long time without spoiling.

Typically, a storage warehouse capable of storing items such as food may include a refrigerator for refrigerated storage of the items and a freezer for frozen storage. The storage warehouse can maintain the temperature of storage rooms at a set target temperature by repeatedly performing a cooling cycle including compression, condensation, expansion, and evaporation of a refrigerant. In other words, the storage warehouse supplies air cooled by an evaporator provided corresponding to each storage room (the refrigerator and/or the freezer) based on the target temperature of each storage room into each storage room so that the temperature of the storage room is maintained at the target temperature.

Recently, refrigerators have evolved to provide a graphical user interface to the user through a display for indicating the temperature of the storage room and the operation mode of the refrigerator, allowing the user to easily obtain information related to the refrigerator and/or information related to food.

However, food items stored in the storage warehouse may exhibit different quality states even in the same storage environment depending on their type or size. For example, in the case of certain fruits, relatively small fruits may maintain freshness better than relatively large fruits in the same environment. In addition, the form of changes in the freshness of fruits may differ depending on the type, production area, or cultivation period of the fruit. Thus, appropriate management is required for each item stored in the storage warehouse, but it is too difficult for a manager to manually check all stored items, and the accuracy also varies from person to person, making it difficult to manage stored items, especially food items.

In addition, among foods stored in the storage warehouse, in the case of foods that require ripening, it is often not easy to check the degree of ripening. In particular, the experience of an expert is needed to accurately check the degree of ripening, and even experts may judge the degree of ripening differently depending on each individual's condition or situation. In addition, even if the same storage warehouse and storage environment are provided, the ripening completion date of the food may differ depending on the state of the food at the time of being received in the storage warehouse for ripening or the storage environment of the storage warehouse. Because of this, even if the food is received into the storage warehouse and shipped out after a specified period of time, there is a problem in which the degree of ripening of the food is not uniform.

### SUMMARY

The present invention is intended to provide a food state tracking method based on a spectral image, as well as a food management device supporting the same, which collects and observes spectral images of food during the process of storing and preserving the food, provides food state tracking information, and allows the freshness of the food to be appropriately maintained based on the information.

The present invention is also intended to provide a food management method based on a spectral image, as well as a device supporting the same, which can provide a stable and uniform food ripening function by making it possible to uniformly achieve a desired degree of ripening in relation to the ripening of food.

However, the objects of the present invention are not limited to the above objects, and other objects not mentioned can be clearly understood from the description below.

To achieve the above objects, a food management device supporting food state tracking based on a spectral image may include at least one spectral camera for acquiring spectral images of food received in a storage warehouse, and a processor functionally connected to the at least one spectral camera. The processor may be configured to receive food-related information of the received food from the storage warehouse, to acquire the spectral images of the food at regular intervals by activating the at least one spectral camera, and to monitor a change in a state of the food by analyzing the acquired spectral images.

Specifically, the processor may be configured to identify a location of the received food from the food-related information, and to activate the spectral camera disposed at the location of the food.

Specifically, the processor may be configured to deactivate the spectral camera disposed at the location when receiving a message regarding shipment of the food from the storage warehouse.

Specifically, the processor may be configured to acquire an initial spectral image at a time the food is received, and to track the change in the state of the food by comparing the initial spectral image with the spectral images acquired at regular intervals thereafter.

Specifically, the processor may be configured to, if the change in the state of the food is greater than or equal to a predefined reference value, notify the change in the state of the food to a designated terminal.

Specifically, the processor may be configured to, if the change in the state of the food is greater than or equal to a predefined reference value, collect information on a storage environment corresponding to the changed state of the food, create change information for changing a storage environment of the food based on the collected information, and transmit the change information to the storage warehouse.

Specifically, the processor may be configured to create the change information for changing at least one of temperature, humidity, and light amount in the storage environment of the food.

A food state tracking method based on a spectral image according to an embodiment of the present invention may include, by a processor of a food management device that manages storage of food, establishing a communication channel with a storage warehouse where the food is stored, receiving food-related information on the food received in the storage warehouse from the storage warehouse, acquiring spectral images of the food at regular intervals by using a spectral camera disposed to capture the spectral images of the food, and monitoring a change in a state of the food by analyzing the acquired spectral images.

Specifically, acquiring spectral images may include identifying a location of the received food from the food-related information, and activating the spectral camera disposed at the location of the food.

Specifically, the method may further include receiving a message regarding shipment of the food from the storage warehouse, and deactivating the spectral camera disposed at the location.

Specifically, monitoring a change in a state of the food may include at least one of, if the change in the state of the food is greater than or equal to a predefined reference value, notifying the change in the state of the food to a designated terminal, and if the change in the state of the food is greater than or equal to a predefined reference value, collecting information on a storage environment corresponding to the changed state of the food, creating change information for changing a storage environment of the food based on the collected information, and transmitting the change information to the storage warehouse.

In addition, a food management device supporting a food management function based on a spectral image according to the present invention may include at least one spectral camera for acquiring spectral images of a ripened food received in a storage warehouse, and a processor functionally connected to the at least one spectral camera. The processor may be configured to, when the ripened food is received at a point of the storage warehouse, activate a spectral camera disposed to photograph the point of the storage warehouse, to acquire a spectral image of the ripened food through the activated spectral camera, to detect a ripening state of the ripened food by comparing the spectral image with a pre-stored reference model, and to output a message according to the ripening state.

Specifically, the processor may be configured to check a current degree of ripening through comparison between the detected ripening state and the reference model, and if the degree of ripening is within a target reference range, to output a message based on completion of ripening.

Specifically, the processor may be configured to, if the degree of ripening is below the reference range, output a message including at least one of a message indicating an underripe state and a remaining time until a time of the ripening completion.

Specifically, the processor may be configured to, if a storage environment of the storage warehouse needs to be changed based on the underripe state, create change information related to the change of the storage environment and transmit the change information to the storage warehouse.

Specifically, the processor may be configured to, if the degree of ripening exceeds the reference range, output a message indicating an overripe state.

A food management method based on a spectral image according to an embodiment of the present invention may include, by a processor of a food management device, receiving food-related information related to storage of a ripened food from a storage warehouse, activating a spectral camera disposed to photograph a point of the storage warehouse, based on the food-related information, acquiring a spectral image of the ripened food through the activated spectral camera, detecting a ripening state of the ripened food by comparing the spectral image with a pre-stored reference model, and outputting a message according to the ripening state.

Specifically, detecting a ripening state of the ripened food may include checking a current degree of ripening through comparison between the detected ripening state and the reference model, and if the degree of ripening is within a target reference range, outputting a message based on completion of ripening, if the degree of ripening is below the reference range, outputting a message including at least one of a message indicating an underripe state and a remaining time until a time of the ripening completion, or if the degree of ripening exceeds the reference range, outputting a message indicating an overripe state.

Specifically, the method may further include, if a storage environment of the storage warehouse needs to be changed based on the underripe state, creating change information related to the change of the storage environment and transmitting the change information to the storage warehouse.

A food management device supporting a food management function based on a spectral image according to an embodiment of the present invention may include a server communication circuit establishing a communication channel with a storage warehouse and a food management device, and a server processor functionally connected to the server communication circuit. The server processor may be configured to, when receiving a message about reception of a ripened food from the storage warehouse, request a spectral image of the ripened food from the food management device that controls the spectral camera disposed at a location where the ripened food is received, when receiving the spectral image from the food management device, detect a ripening state of the ripened food by comparing the spectral image with a pre-stored reference model, and output a message based on the detected ripening state.

Specifically, the server processor may be configured to check a current degree of ripening through comparison between the detected ripening state and the reference model, and if the degree of ripening is within a target reference range, to output a message based on completion of ripening, if the degree of ripening is below the reference range, to output a message including at least one of a message indicating an underripe state and a remaining time until a time of the ripening completion, or if the degree of ripening exceeds the reference range, to output a message indicating an overripe state.

According to the present invention, the food state tracking method based on spectral images and the food management device supporting the same can easily support food state tracking based on spectral images of food, and also support more efficient and appropriate food management based on the food state tracking.

Further, according to the present invention, the food management method based on spectral images and the device supporting the same can systematize checking the degree of ripening of food based on spectral images, and also support uniform ripening management and accurate shipping management of food.

In addition, various effects other than the effects described above can be directly or implicitly disclosed in the detailed description according to embodiments of the present invention to be described later.

### BRIEF DESCRIPTION OF DRAWINGS

FIG. 1 is a diagram showing an example of a system environment supporting a food state tracking function according to the first embodiment of the present invention.

FIG. 2 is a diagram showing an example of components of a storage warehouse capable of storing food according to the first embodiment of the present invention.

FIG. 3 is a diagram showing an example of components of a food management device according to the first embodiment of the present invention.

FIG. 4 is a diagram showing an example of components of a processor among the components of the food management device according to the first embodiment of the present invention.

FIG. 5 is a diagram showing an example of components of a server device according to the first embodiment of the present invention.

FIG. 6 is a diagram showing an example of a food state tracking method of a food management device according to the first embodiment of the present invention.

FIG. 7 is a diagram showing an example of a food management method based on food state tracking according to the first embodiment of the present invention.

FIG. 8 is a diagram showing an example of a system environment supporting a food management function according to the second embodiment of the present invention.

FIG. 9 is a diagram showing an example of components of a storage warehouse capable of storing food according to the second embodiment of the present invention.

FIG. 10 is a diagram showing an example of components of a food management device according to the second embodiment of the present invention.

FIG. 11 is a diagram showing an example of components of a processor among the components of the food management device according to the second embodiment of the present invention.

FIG. 12 is a diagram showing an example of components of a server device according to the second embodiment of the present invention.

FIG. 13 is a diagram showing an example of a food management method of a food management device according to the second embodiment of the present invention.

FIG. 14 is a diagram showing an example of server device operation related to the food management method according to the second embodiment of the present invention.

### DETAILED DESCRIPTION OF EMBODIMENTS

Now, embodiments of the present invention will be described in detail with reference to the accompanying drawings.

However, in the following description and the accompanying drawings, well known functions and components may not be described nor illustrated in detail to avoid obscuring the subject matter of the present invention. In addition, identical components are indicated with the same reference numerals as much as possible throughout the drawings

The terms or words used in the following description and drawings should not be interpreted as limited to their usual or dictionary meanings and should be interpreted as meanings and concepts that conform to the technical idea of the present invention based on the principle that the inventor can appropriately define the concept of the terms to best describe his or her invention. Therefore, embodiments described herein are only the most preferred embodiments of the present invention and do not represent all of the technical ideas of the present invention. Thus, it should be understood that there may be various equivalents and modified examples that can replace the embodiments at the time of filing this application.

In addition, terms including ordinal numbers such as first, second, etc. are used to describe various elements only for the purpose of distinguishing one element from another, and are not used to limit such elements. For example, without departing from the scope of the present invention, a second element may be named a first element, and similarly, a first element may also be named a second element.

In addition, terms used herein are only for describing specific embodiments and do not limit the present disclosure. The singular expression includes the plural expression unless the context clearly indicates otherwise. Also, the terms such as “comprise” and “include” used herein are intended to specify the presence of features, numerals, steps, operations, elements, components, or combinations thereof, which are disclosed herein, and should not be construed to exclude in advance the possibility of the presence or addition of other features, numerals, steps, operations, elements, components, or combinations thereof.

In addition, the terms such as “unit” and “module” used herein refer to a unit that processes at least one function or operation and may be implemented with hardware, software, or a combination of hardware and software. In addition, the terms “a”, “an”, “one”, “the”, and similar terms may be used as both singular and plural meanings in the context of describing the present invention (especially in the context of the following claims) unless the context clearly indicates otherwise.

In addition to the terms mentioned above, specific terms used in the following description are provided to help understanding of the present invention, and the use of such specific terms may be changed to other forms without departing from the technical meaning of the present invention.

Also, embodiments within the scope of the present invention include computer-readable media having computer-executable instructions or data structures stored on computer-readable media. Such computer-readable media can be any available media that is accessible by a general purpose or special purpose computer system. By way of example, such computer-readable media may include, but not limited to, RAM, ROM, EPROM, CD-ROM or other optical disk storage, magnetic disk storage or other magnetic storage devices, or any other physical storage medium that can be used to store or deliver certain program codes formed of computer-executable instructions, computer-readable instructions or data structures and which can be accessed by a general purpose or special purpose computer system.

### First Embodiment

Hereinafter, in the present invention, a system environment supporting a food state tracking function and a food management function based thereon, and the types and roles of each component included therein will be described.

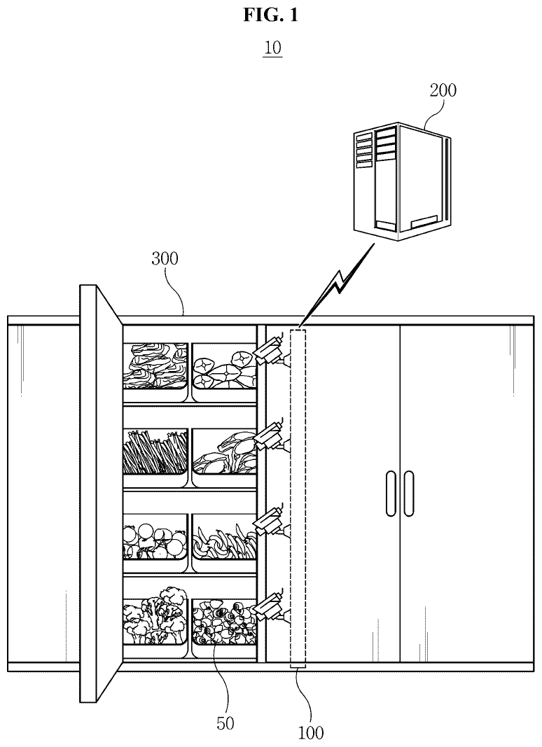

FIG. 1 is a diagram showing an example of a system environment supporting a food state tracking function according to the first embodiment of the present invention.

Referring to FIG. 1, the system environment 10 of the present invention that supports the food state tracking function and the state management may include at least one food 50 (e.g., fruit, vegetable, processed food, etc.), a storage warehouse 300 in which the food 50 is stored, a food management device 100, and a server device 200 capable of supporting the food management device 100. In an example of the system environment 10, a communication channel is formed between the food management device 100 and the server device 200, and the server device 200 supports the food management device 100. However, the present invention is not limited thereto. For example, the food management device 100 may be configured to support the state tracking and storage management for the food 50 based on an embedded program without the server device 200. In this case, the server device 200 may be omitted from the system environment 10.

The at least one food 50 may include at least one of various agricultural, livestock and fishery products that can be stored in the storage warehouse 300. For example, as stored products that can be stored in the storage warehouse 300, the food 50 may include at least one type of agricultural product, fishery product, and livestock product, or at least one type of processed agricultural product food, processed fishery product food, and processed livestock product food. In an example, the food 50 may include fruits such as strawberries, melons, watermelons, apples, pears, bananas, and tangerines, grains such as rice or barley, and the like. In addition, the food 50 may include various fish, shellfish, crustaceans, and the like. The food 50 may be stored in different forms depending on its type, and even if it is the same type, the stored form may be different depending on its size or shape. Therefore, the food 50 may be stored in different storage environments according to at least one of the criteria of type, size, and shape in the storage warehouse 300.

The storage warehouse 300 may have at least one space where the food 50 can be stored. The storage warehouse 300 may be built in a fixed form at a designated location or in a mobile form. The storage warehouse 300 may be built in a form where a plurality of foods 50 can be stored. In this case, the storage warehouse 300 may be provided with partitions or isolated from adjacent spaces so that the food 50 can be stored separately by type, size, or shape. The external shape or construction material of the storage warehouse 300 may vary. For example, the storage warehouse 300 may be provided in a cuboid shape, a dome shape, or an underground facility. The storage warehouse 300 may control the storage environment (e.g., temperature, humidity, light, etc.) to maintain and manage the stored food 50 under the control of the food management device 100 or the server device 200.

The food management device 100 may be arranged to obtain a spectral image of the at least one food 50. For example, the food management device 100 may collect spectral images of the food 50 by using at least one spectral camera arranged at a certain point in the storage warehouse 300 where the at least one food 50 is arranged. In this regard, the spectral camera may be arranged to photograph the food 50 stored at a certain point (e.g., a shelf) in the storage warehouse 300. The food management device 100 may activate the arranged spectral camera according to predefined schedule information or a specified time lapse under a specified storage environment, acquire a plurality of spectral images of the food 50 by using the activated spectral camera, and perform an analysis on the acquired plurality of spectral images to track the state of the food 50. Additionally, the food management device 100 may collect storage management information regarding the state-specific management of the food 50 through the server device 200. The food management device 100 may transmit control information for controlling the storage environment of the corresponding food 50 to the storage warehouse 300 according to the state-specific storage management information of the food 50. Upon receiving the control information, the storage warehouse 300 may adjust at least some elements (e.g., temperature, humidity, light) of the storage environment. If the change in the state of the stored food 50 exceeds a predefined reference value, the food management device 100 may output guidance information about this.

The server device 200 can establish a communication channel with the food management device 100. The server device 200 may receive information related to the food 50, such as at least one spectral image taken of the food 50 and information on the storage environment of the food 50, from the food management device 100, generate state guidance information for the food 50 or control information for changing the storage environment of the food 50 according to the received information, and transmit the generated information to the storage warehouse 300 directly or through the food management device 100. Here, the server device 200 is a component that receives spectral images related to the food 50 from the food management device 100 and analyzes them, and it may be omitted if the food management device 100 is designed to directly perform state analysis and storage environment control of the food 50.

As described above, the system environment 10 supporting the food state tracking and management function according to the first embodiment of the present invention can obtain spectral images related to the food 50 by placing the food management device 100 configured to obtain the spectral images of the food 50 in the storage warehouse 300 where the food 50 is stored. In the system environment 10, the food management device 100 can perform state tracking of the food 50 by performing analysis on the obtained spectral images, and also support the freshness of the food 50 to be properly maintained for a long time by applying state-specific storage management of the food 50.

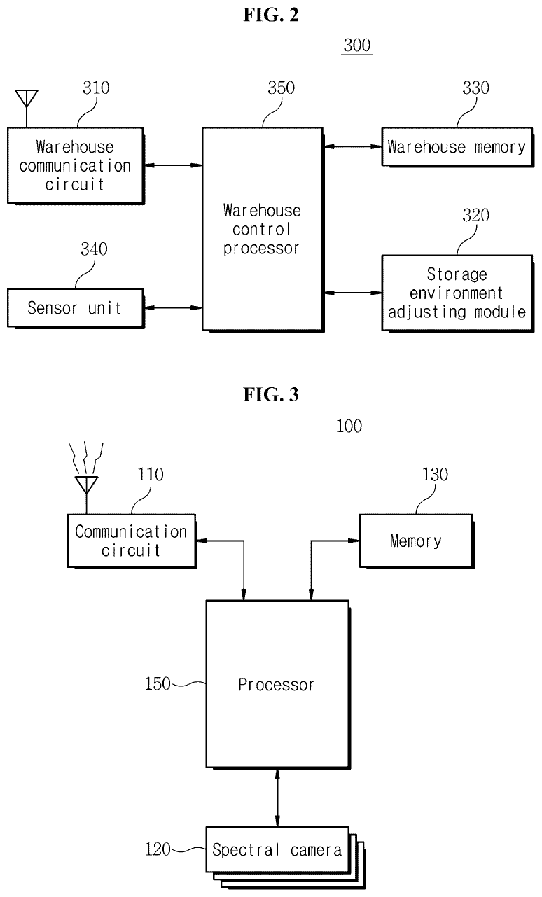

FIG. 2 is a diagram showing an example of components of a storage warehouse capable of storing food according to the first embodiment of the present invention.

Referring to FIG. 2, the storage warehouse 300 may include a warehouse communication circuit 310, a storage environment adjusting module 320, a warehouse memory 330, a sensor unit 340, and a warehouse control processor 350.

The warehouse communication circuit 310 may form a communication channel of the storage warehouse 300. For example, the warehouse communication circuit 310 may form a communication channel with at least one of the food management device 100 and the server device 200 and transmit operation information related to the operation of the storage warehouse 300 to the food management device 100 or the server device 200. The operation information of the storage warehouse 300 may include at least some of, for example, the type of the food 50 being stored, information on the storage environment for each food 50, the storage date of the food 50, and the expected shipping date of the food 50. The warehouse communication circuit 310 may receive storage environment change information (e.g., setting information for changing the states of the storage environment) related to the food 50 from the food management device 100 or the server device 200.

The storage environment adjusting module 320 may include a module that adjusts the environment of at least some areas of the storage warehouse 300 or an area where the food 50 is stored. For example, the storage environment adjusting module 320 may include at least one of a temperature adjusting device capable of adjusting the temperature of a certain area where the food 50 is placed, a humidity adjusting device capable of adjusting humidity, and a light irradiation device capable of adjusting the amount of light irradiation. The storage environment adjusting module 320 may adjust at least one of temperature, humidity, and light to maintain the freshness of the food 50 for a long time under the control of the warehouse control processor 350.

The warehouse memory 330 may store various data and programs required for the operation of the storage warehouse 300. For example, the warehouse memory 330 may include a program for operating and controlling the at least one adjusting device included in the storage environment adjusting module 320. In addition, the warehouse memory 330 can store at least one program for operating and controlling the sensor unit 340 arranged in the storage warehouse 300. The warehouse memory 330 may store information on the type of the food 50 that is received in the storage warehouse 300, the received date of the food 50, the location information in the storage warehouse 300 where the food 50 is placed, the number of days of storage of the food 50, the expected shipping date of the food 50, etc. In addition, the warehouse memory 330 may store the current storage environment setting information of the food 50.

The sensor unit 340 may be placed in at least some areas of the storage warehouse 300 and collect various kinds of sensor information related to the food 50. For example, the sensor unit 340 may include at least one of a location detection sensor capable of identifying a location where the food 50 is placed, and a sensor (e.g., at least one of a temperature sensor, a humidity sensor, and a light detection sensor) capable of sensing a storage environment where the food 50 is placed. The sensor unit 340 may collect sensing information on an area where the food 50 is placed under the control of the warehouse control processor 350, and transmit the collected sensing information to the warehouse control processor 350.

The warehouse control processor 350 may perform various operation controls required for operating the storage warehouse 300. For example, the warehouse control processor 350 may adjust at least some of the temperature, humidity, and light amount of a specific area of the storage warehouse 300 according to the storage warehouse setting information. In this operation, the warehouse control processor 350 may control the sensor unit 340 to sense the temperature, humidity, and light amount of a specific area where the food 50 is placed, and adjust the temperature, humidity, and light amount to correspond to the values set in the setting information. In an example, when the food 50 is received and the storage warehouse setting information is determined, the warehouse control processor 350 may transmit the type of the food 50, the location information where the food 50 is placed, the expected shipping date of the food 50, and the current storage environment setting information of the area where the food 50 is placed to the food management device 100 or the server device 200. Upon receiving storage environment change information from the food management device 100 or the server device 200, the warehouse control processor 350 may adjust at least some of the temperature, humidity, and light amount of the storage environment in response to the received storage environment change information. When the food 50 is shipped out, the warehouse control processor 350 may transmit information on the shipping of the food 50 to the food management device 100 or the server device 200, and control the sensor unit 340 and the storage environment adjusting module 320 at the area where the food 50 is placed to be turned off.

FIG. 3 is a diagram showing an example of components of a food management device according to the first embodiment of the present invention, and FIG. 4 is a diagram showing an example of components of a processor among the components of the food management device according to the first embodiment of the present invention.

First, referring to FIG. 3, the food management device 100 according to the first embodiment of the present invention may include a communication circuit 110, a spectral camera 120, a memory 130, and a processor 150. Additionally, the food management device 100 may further include a mounting structure for mounting the spectral camera 120 so that the spectral camera 120 can photograph an area where at least one food 50 is placed. In addition, the food management device 100 may further include a power supply (e.g., a permanent power supply or a battery) required for the operation of at least one of the above-mentioned components, for example, the communication circuit 110, the spectral camera 120, the memory 130, and the processor 150.

The communication circuit 110 can establish a communication channel with the storage warehouse 300 or the server device 200. If the server device 200 is designed to perform the calculation required for the food state tracking and management function according to the first embodiment of the present invention, the communication circuit 110 may transmit at least one spectral image collected by the spectral camera 120 to the server device 200. On the other hand, the food state tracking and management function may be performed independently by the food management device 100. In this case, the communication circuit 110 may directly transmit a manager notification message or user notification message generated during the operation of the food state tracking and management function to a manager's or user's terminal device. Alternatively, the communication circuit 110 may transmit a notification message to the server device 200 in response to the control of the processor 150.

The communication circuit 110 can receive information related to the storage of the food 50 from the storage warehouse 300 (e.g., information on the type of the food 50 received in the storage warehouse 300, the received date of the food 50, location information in the storage warehouse 300 where the food 50 is placed, the number of days of storage of the food 50, the expected shipping date of the food 50, etc.). The communication circuit 110 may transmit change information related to a change in the storage environment of the food 50 to the storage warehouse 300 in response to the control of the processor 150.

The spectral camera 120 may be arranged to capture a spectral image of at least one food 50. Alternatively, a plurality of spectral cameras 120 may be arranged in a plurality of areas of the storage warehouse 300 to capture spectral images of the food 50 located within the storage warehouse 300. In an example, when the food 50 is stored at a certain location (e.g., a food rack where the food 50 can be placed) in the storage warehouse 300, the spectral camera 120 may collect spectral images of the food 50 under the control of the processor 150. In addition, the spectral camera 120 may collect spectral images of the food 50 over time. Also, the spectral camera 120 may collect spectral images under the control of the processor 150 when states of the storage environment change or when the storage environment (e.g., at least one of temperature, humidity, and light amount) changes.

The memory 130 can store at least one program or data required for the operation of the food management device 100. For example, the memory 130 may store a control program required for operating the spectral camera 120 and a plurality of spectral images acquired through the spectral camera 120. For example, the memory 130 may store a spectral image captured when the food 50 is received in the storage warehouse 300, spectral images captured every time a specified period of time has elapsed since the food 50 was received, and spectral images captured every time the storage environment is changed.

The processor 150 may perform at least one of the transmission and processing of signals required for the operation of the food management device 100 and the storage and output of processing results. For example, the processor 150 may control the acquisition of a spectral image related to food 50 using the spectral camera 120 in response to at least one of a predefined event, guidance information from the storage warehouse 300 (e.g., information notifying a situation in which new food 50 has arrived, information notifying that the storage environment of the food 50 has changed), or a request from the server device 200. The processor 150 may track the state of the food 50 based on at least one acquired spectral image and perform output of designated information according to the change in state. In this regard, the processor 150 may include components as illustrated in FIG. 4.

Referring to FIG. 4, the processor 150 may include a spectral image collector 151, a spectral image analyzer 152, a food state notifier 153, and a warehouse controller 154.

The spectral image collector 151 can control acquisition of a spectral image using the spectral camera 120. For example, upon receiving guidance information (or a message) regarding the arrival of new food 50 from the storage warehouse 300, the spectral image collector 151 may process acquisition of a spectral image for the newly arrived food 50. In this process, the spectral image collector 151 may receive location information of the food 50 from the storage warehouse 300 and control the spectral camera 120 placed at the corresponding location to acquire an initial spectral image for the food 50. Thereafter, the spectral image collector 151 may periodically acquire spectral images for the food 50 over a specified period of time. Alternatively, the spectral image collector 151 may control the collection of spectral images for the food 50 in response to a request from a manager of the food management device 100 or a request from the server device 200. When the spectral image collector 151 receives from the storage warehouse 300 guidance information (or guidance message) indicating that at least a part of the storage environment of a certain food 50 will be changed, the spectral image collector 151 may acquire a spectral image for the food 50 at the time of the change in the storage environment. When the food 50 at a specific location is shipped out, the spectral image collector 151 may control the spectral camera 120 positioned to photograph the location where the food 50 is placed to be deactivated.

The spectral image analyzer 152 can analyze at least one spectral image collected by the spectral image collector 151 and store the analysis result in the memory 130. For example, the spectral image analyzer 152 may check whether a spectral image in which a change greater than a reference value has occurred is collected based on the initial spectral image. In this process, the spectral image analyzer 152 may perform clustering model learning on a spectrum of the initial spectral image, store the spectrum as a cluster, and compares a spectrum of the subsequently acquired spectral image with the stored cluster to check whether it is out of a reference range (e.g., average value of the cluster+K*standard deviation (K is a constant)). If the centroid of the spectrum of the subsequently acquired spectral image is out of the reference range, identification information corresponding to the spectral image may be transmitted to the food state notifier 153. Next, the spectral image analyzer 152 may set the spectral image that is out of the reference range as a new reference spectral image, and compare a spectral image acquired thereafter with the new reference spectral image to determine whether it is out of a predefined new reference range. As described above, the spectral image analyzer 152 may continuously analyze spectral images of the food 50, and whenever the state of the food 50 changes beyond the reference range, the spectral image analyzer 152 may transmit an alarm about such changes to the food state notifier 153 and the warehouse controller 154.

When food state notifier 153 receives an alarm about a change in the state of the food 50 from the spectral image analyzer 152, it may collect information about the food 50 for which the alarm has occurred. For example, the food state notifier 153 may collect the date of receipt, degree of change, expected shipping date of the food 50 for which the alarm has occurred, etc., and transmit the collected information related to the food 50 to a terminal device of a manager of the food management device 100 or a designated user. Alternatively, the food state notifier 153 may output the information related to the food 50 for which the alarm has occurred through an output device. In this regard, the food management device 100 may include at least one of a display or an audio output device. The manager may identify the storage state of the food 50 by checking the information about the food 50 that has changed beyond a predefined reference range through the output device such as the display, and adjust the storage environment or expected shipping date.

The warehouse controller 154 can control the change of the storage environment for the food 50 for which an alarm has received from the spectral image analyzer 152. In this regard, the warehouse controller 154 may collect storage environment setting information for each state of a specific food 50. For example, the warehouse controller 154 may identify the current state of the food 50 and the expected shipping date of the food 50 and determine the storage environment setting information (e.g., at least one of temperature, humidity, and light intensity) for optimally maintaining the current state before the expected shipping date of the food 50. In this regard, information for maintaining the freshness of the food 50 for each state may be acquired from a designated external server device. Alternatively, the food management device 100 may store and provide a database of setting information to be changed for maintaining freshness according to a change in the state of the food 50 in the memory 130. The warehouse controller 154 may obtain new storage environment change information according to the change in the state of the food 50 and provide the storage environment change information to the storage warehouse 300. Based on the received storage environment change information, the storage warehouse 300 may change the storage environment of the food 50 whose state has changed. For example, the storage warehouse 300 may increase the humidity of the food 50 and reduce the amount of light compared to the previous storage environment, based on the storage environment change information. Meanwhile, the storage environment change information may vary depending on the type of the food 50 or the state of the food 50, and such information may be accumulated and stored empirically or acquired from a designated external server device. The storage management information of the food 50 accumulated and stored empirically may be used to better maintain the freshness of the food 50 when the same food 50 is stored.

As described above, the food management device 100 according to the first embodiment of the present invention can support monitoring of changes in the state of the food 50 by collecting and analyzing spectral images of the food 50 disposed and stored at one or more locations of the storage warehouse 300 over time or with the occurrence of an event. Accordingly, the food management device 100 of the present invention can support predicting the state of the food in the future and optimizing inventory management based on food monitoring data over time. In addition, the food management device 100 of the present invention can support better maintenance of the freshness of the food 50 by providing control information for a more optimal storage environment according to changes in the state of the food 50. Additionally, when the change in the state of a specific food 50 exceeds a predefined risk criterion, the food management device 100 can suggest disposal or immediate shipment of the food 50 to reduce resources required for storing unnecessary food 50 and prevent damage to other foods caused by spoiled food 50. For example, when the food is determined to be already spoiled based on spectral image analysis, the food management device 100 according to the first embodiment of the present invention can backtrack the previous state (calculate an estimated value in reverse order of time flow) so that it can be used to explore future management measures for the product.

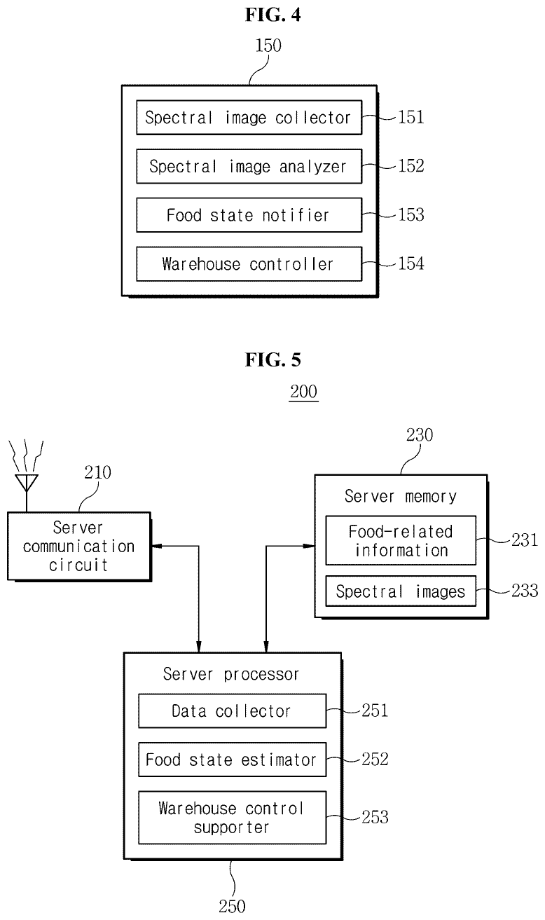

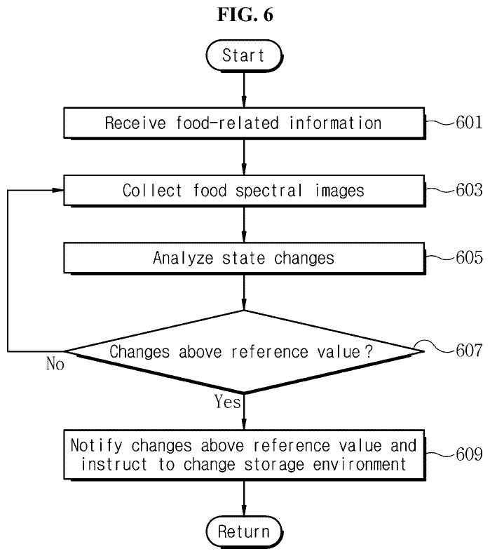

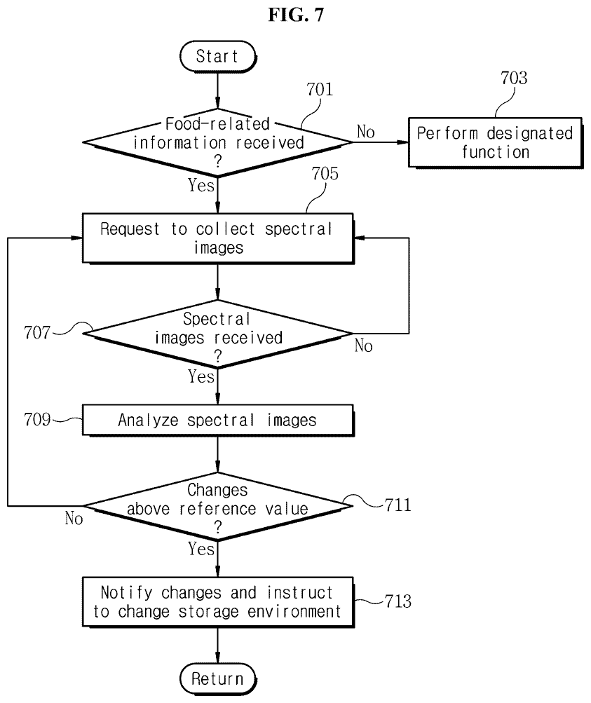

Meanwhile, in the above description, the food management device 100 is described as monitoring the state of the food 50 stored in the storage warehouse 300 and directly controlling the change in the storage environment according to the change in the state of the food 50, but the present invention is not limited thereto. For example, the food management device 100 may be designed to control only the spectral camera 120 to collect spectral images of the food 50 and transmit the collected spectral images to the server device 200. In this case, the server device 200 may support a function of monitoring and managing the state of the food 50 based on the components as illustrated in FIG. 5.

FIG. 5 is a diagram showing an example of components of a server device according to the first embodiment of the present invention. As described above, if the food management device 100 is designed to perform the food state tracking function and the food management function according to the first embodiment of the present invention, the server device 200 may be omitted.

Referring to FIG. 5, the server device 200 of the present invention may include a server communication circuit 210, a server memory 230, and a server processor 250.

The server communication circuit 210 may directly form a communication channel with the food management device 100. Additionally, the server communication circuit 210 may form a communication channel with the storage warehouse 300 or communicate with the storage warehouse 300 via the food management device 100. The server communication circuit 210 may receive at least one spectral image from the food management device 100 in response to a specified period or occurrence of a predefined event. Additionally, the server communication circuit 210 may receive at least one food-related information from the storage warehouse 300. The server communication circuit 210 may transmit storage environment change information to the storage warehouse 300 in response to control of the server processor 250.

The server memory 230 can store at least one program or data required for the operation of the server device 200. For example, the server memory 230 may store at least one food-related information 231 received from the storage warehouse 300. In addition, the server memory 230 may store a plurality of spectral images 233 related to a specific food 50 provided by the food management device 100.

The server processor 250 can control the transmission and processing of signals required for operating the server device 200, storage or transmission of results, or transmission of messages corresponding to results. In this regard, the server processor 250 can include a data collector 251, a food state estimator 252, and a warehouse control supporter 253.

The data collector 251 can receive food-related information from the storage warehouse 300. In this regard, the data collector 251 may form a communication channel with the storage warehouse 300, and when new food 50 is received in the storage warehouse 300 or food 50 being stored is shipped out, the data collector 251 may receive food-related information from the storage warehouse 300. The data collector 251 may temporarily or semi-permanently store the received food-related information 231 in the server memory 230. Upon receiving the food-related information from the storage warehouse 300, the data collector 251 may request a spectral image corresponding to the food from the food management device 100. The data collector 251 may receive, from the food management device 100, spectral images captured at regular time intervals from the initial time when the food 50 is placed in the storage warehouse 300. The spectral images 233 captured regarding the food 50 may be stored in the server memory 230. The operation of collecting the spectral images 233 and storing them in the server memory 230 may be repeatedly performed at regular intervals until the food 50 is shipped out from the storage warehouse 300 or until a request is made by a manager of the food management device 100 or of the server device 200.

The food state estimator 252 can analyze the initial spectral image received from the food management device 100 at the time when the food-related information 231 is collected, and thereby determine the freshness of the corresponding food 50. For example, the food state estimator 252 may compare the spectrum of the initial spectral image of the food 50 with the spectrum of the spectral image when the corresponding food 50 has the optimal freshness, and thereby determine the freshness of the food 50 stored in the storage warehouse 300. The spectral image of the food 50 with the optimal freshness may be received from a designated external server device or designated by a manager of the server device 200. The food state estimator 252 may analyze the spectral images 233 acquired at regular intervals to determine how much change occurs from the initial spectral image, and estimate the change in freshness of the corresponding food 50. If analysis of the spectral images 233 indicates that the state of the food 50 is changed to a predefined reference value or more, the food state estimator 252 may output an alarm. This alarm may be transmitted to at least one of a terminal of a manager of the food management device 100, a terminal of an operator of the server device 200, and a terminal of a manager of the storage warehouse 300. Additionally, when the food management device 100 includes a separate display device, the food state estimator 252 may output the alarm through the display of the food management device 100. In addition, when the state of the food 50 is changed to a predefined value or more, the food state estimator 252 may transmit the corresponding change to the warehouse control supporter 253.

Upon receiving the state change regarding the food 50 from the food state estimator 252, the warehouse control supporter 253 may create storage environment change information regarding the corresponding food 50 and transmit the created storage environment change information to the storage warehouse 300. In this regard, the server device 200 may store and manage a table that stores storage environment setting information according to the state of the food 50. The warehouse control supporter 253 may identify the food-related information 231, identify environmental factors (e.g., at least one of temperature, humidity, and light intensity) that need to be changed according to the current state of the food 50, create storage environment change information requesting a change in the corresponding environmental factors, and transmit the storage environment change information to the storage warehouse 300.

FIG. 6 is a diagram showing an example of a food state tracking method of a food management device according to the first embodiment of the present invention.

Referring to FIG. 6, in a method of operating the food management device 100 related to the food state tracking method according to the first embodiment of the present invention, the processor 150 of the food management device 100 may receive food-related information in step 601. In this regard, the food management device 100 may establish a communication channel with the storage warehouse 300, and when new food 50 is received in the storage warehouse 300, the storage warehouse 300 may transmit the food-related information to the food management device 100. Here, the food-related information may include at least some of the name of the food 50, the received date of the food 50, the placed location of the food 50, and the expected shipping date of the food 50.

When the processor 150 of the food management device 100 receives the food-related information, it may collect spectral images of the food 50 in step 603. In this process, the food management device 100 may check the food-related information to identify the location of the food 50, identify the spectral camera 120 disposed to be able to photograph the corresponding location, and collect spectral images by using the spectral camera 120 at regular intervals from the time the food 50 is received. In relation to this function, the food management device 100 may store and manage location information about a plurality of spectral cameras disposed to photograph a plurality of locations where the food 50 can be placed in the storage warehouse 300, and about an area that each of the spectral cameras can photograph.

In step 605, the processor 150 of the food management device 100 may perform an analysis on the collected spectral images to analyze changes in the state of the food 50. For example, the processor 150 may perform a spectral comparison between the spectral image obtained at the time the food 50 was initially received and the spectral image obtained at the current time. Alternatively, the processor 150 may perform a spectral comparison between the spectral image captured of the food 50 having a predetermined specific freshness and the spectral image obtained at the current time.

In step 607, the processor 150 may determine whether the spectrum comparison result is greater than or equal to a reference value. If it is less than the reference value, the processor 150 may return to step 603 and perform the food spectral image collection. If the spectrum comparison result shows a change greater than or equal to the reference value, the processor 150 may perform in step 609 notifying the change above the reference value and instructing to change the storage environment. Alternatively, the processor 150 may perform only notifying the change above the reference value or perform only instructing to change the storage environment. This notification of the change above the reference value may be output to, for example, a terminal of a manager of the storage warehouse 300, a display device connected to the food management device 100, or a terminal of a manager of the food management device 100. In relation to changing the storage environment, the processor 150 may generate control information including a value of an environmental factor (e.g., at least one of temperature, humidity, and light intensity) for changing the storage environment, and transmit the control information to the storage warehouse 300.

Next, the food management device 100 may return to step 603 and re-perform the subsequent operations. Through this, the food management device 100 can adaptively change the storage environment as the state of the food 50 changes, thereby supporting the freshness of the food 50 to be maintained within a certain range for a long time. Meanwhile, when new food 50 is received, the food management device 100 may return to step 601 and re-perform the subsequent operations. In addition, the food management device 100 may repeatedly re-perform the operations 603 to 609 described above until the time when the corresponding food 50 is shipped out.

FIG. 7 is a diagram showing an example of a food management method based on food state tracking according to the first embodiment of the present invention.

Referring to FIG. 7, in step 701, the server processor 250 of the server device 200 may check whether the food-related information is received. In this regard, the server device 200 may maintain a communication channel with the storage warehouse 300. If the food-related information is not received, the server processor 250 may perform a designated function in step 703. For example, the server processor 250 may perform management of the food 50 previously registered for food monitoring.

When the food-related information is received, the server processor 250 may request spectral image collection in step 705. In this regard, the server device 200 may form a communication channel with the food management device 100 and request the food management device 100 to collect spectral images corresponding to the food-related information received. In this operation, the server device 200 may provide the food management device 100 with information on the location of the food 50 in the storage warehouse 300. The food management device 100 may identify a spectral camera mapped to the location information of the food 50 in the storage warehouse 300, activate the identified spectral camera to acquire spectral images of newly received food 50, and transmit the acquired spectral images to the server device 200.

In step 707, the server processor 250 may check whether a spectral image is received, and if no spectral image is received within a specified time, the server processor 250 may re-perform step 705. Meanwhile, the server processor 250 may request the collection of spectral images more than a predefined number of times, and if no spectral image is received in response, determine that a failure has occurred in the food management device 100. Then, the server processor 250 may take related measures (e.g., notify the failure to a management terminal that manages the food management device 100).

When the spectral image is received, the server processor 250 may perform spectral image analysis in step 709. In this regard, the server processor 250 may store an initial spectral image at the time of receipt of the food 50 and perform comparative analysis with the spectral image acquired thereafter based on the initial spectral image. Alternatively, the server processor 250 may acquire and store a reference spectral image of a specific food 50 having a freshness greater than or equal to a predefined value from a designated external server device and perform a spectrum comparison between the stored reference spectral image and the currently acquired spectral image.

In step 711, the server processor 250 may determine whether a change value detected through spectrum comparison analysis is greater than or equal to a reference value. If the spectrum comparison analysis result shows a value less than the reference value, the server processor 250 may return to step 705 and re-perform the subsequent operations. Alternatively, the server processor 250 may return to step 705 and re-perform the subsequent operations after a specified time has elapsed.

If the spectrum comparison analysis result shows a value greater than or equal to the reference value, the server processor 250 may perform at least one of a change notification and a storage environment change instruction in step 713. In relation to the change notification, the server processor 250 may provide text or an image corresponding to the change notification to at least one of the manager terminal of the storage warehouse 300, the display of the food management device 100, or the manager terminal of the food management device 100. Additionally, the server processor 250 may create change information for changing the storage environment to be optimized for the currently changed state according to the change in the state of the food 50, and transmit the created change information to the storage warehouse 300. Additionally, if the server processor 250 of the server device 200 determines that the food has already spoiled based on the spectral image analysis, it may backtrack the previous state (calculate an estimated value in reverse order of time flow) to collect and store information on the time of spoiling and information on storage environment, allowing it to be used to explore future management measures for the corresponding product.

### Second Embodiment

Hereinafter, in the present invention, a system environment supporting a food state detection function and a food management function based thereon, and the types and roles of each component included therein will be described.

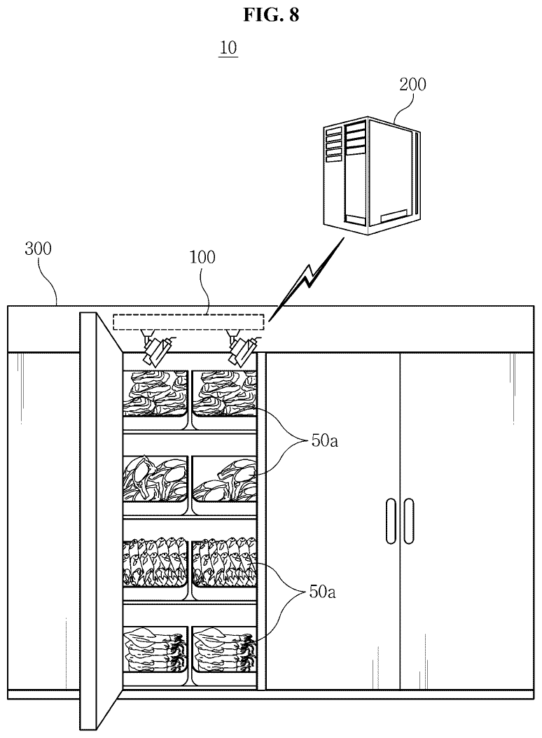

FIG. 8 is a diagram showing an example of a system environment supporting a food management function according to the second embodiment of the present invention.

Referring to FIG. 8, the system environment 10 of the present invention that supports the food management function may include at least one ripened food 50a (e.g., fruit, vegetable, meat, processed food, etc.), a storage warehouse 300 in which the ripened food 50a is stored, a food management device 100, and a server device 200 capable of supporting the food management device 100. In an example of the system environment 10, a communication channel is formed between the food management device 100 and the server device 200, and the server device 200 supports the food management device 100. However, the present invention is not limited thereto. For example, the food management device 100 may be configured to support the state detection, storage management, and shipping management for the ripened food 50a based on an embedded program without the server device 200. In this case, the server device 200 may be omitted from the system environment 10.

The at least one ripened food 50a may include at least one of various agricultural, livestock and fishery products that can be stored in the storage warehouse 300. For example, the ripened food 50a may include, as stored products that can be stored in the storage warehouse 300, at least one type of agricultural product, fishery product, and livestock product, or at least one type of processed agricultural product food, processed fishery product food, and processed livestock product food. In an example, the ripened food 50a may include fruits that require ripening, such as bananas, melons, dragon fruit, tangerines, and mangoes. Additionally, the ripened food 50a may include fish, shellfish, crustaceans, and the like that require ripening. Additionally, the ripened food 50a may include livestock products that require ripening. The ripened food 50a may be stored in different forms and environments depending on its type, and even for the same type, the degree of ripening completion may differ depending on the intended use. Therefore, the ripened food 50a may be stored in different storage environments according to at least one of the criteria of type, purpose, and storage period in the storage warehouse 300.

The storage warehouse 300 may have at least one space of a certain size in which the ripened food 50a can be stored. The storage warehouse 300 may be built in a fixed form at a designated location or in a movable form. The storage warehouse 300 may also be built in a form in which a plurality of ripened foods 50a can be stored. In this case, the storage warehouse 300 may be provided with partitions or isolated from adjacent spaces so that the ripened food 50a can be stored separately by type, purpose, or storage period. The external shape or construction materials of the storage warehouse 300 may vary. For example, the storage warehouse 300 may be provided in a cuboid shape, a dome shape, or an underground facility. This storage warehouse 300 may control the storage environment (e.g., temperature, humidity, light, etc.) to maintain and manage the stored ripened food 50a under the control of the food management device 100 or the server device 200.

The food management device 100 may be arranged to obtain a spectral image of the at least one ripened food 50a. For example, the food management device 100 may collect spectral images of the ripened food 50a by using at least one spectral camera arranged at a certain point in the storage warehouse 300 where the at least one ripened food 50a is arranged. In this regard, the spectral camera may be arranged to photograph the ripened food 50a stored at a certain point (e.g., a shelf) in the storage warehouse 300. The food management device 100 may activate the arranged spectral camera according to predefined schedule information or a specified time lapse under a specified storage environment, acquire at least one spectral image of the ripened food 50a by using the activated spectral camera, and perform an analysis on the acquired at least one spectral image to detect the ripening state of the ripened food 50a. Through this, the food management device 100 may provide information on whether the target ripening degree of the ripened food 50a has been completed, the degree of ripening progress, and the remaining time additionally required for ripening.

Additionally, the food management device 100 may collect setting information on the storage environment (e.g., at least one of temperature, humidity, and light intensity) required for management according to the type, purpose, and storage period of the ripened food 50a through the server device 200. The food management device 100 may transmit control information for controlling the storage environment of the ripened food 50a to the storage warehouse 300 according to the storage environment setting information for each ripened food 50a. For example, in the case of a specific ripened food 50a, there are cases where the ripening environment has to be set differently depending on the degree of ripening. In this case, the food management device 100 may detect the ripening state of the ripened food 50a, generate control information for controlling the ripening environment depending on the ripening state (e.g., information for adjusting at least some of the temperature, humidity, and light intensity of the storage environment), and transmit the control information to the storage warehouse 300.

The server device 200 can establish a communication channel with the food management device 100. The server device 200 may provide the food management device 100 with setting information for adjusting necessary storage environment according to the type of ripened food 50a or the purpose or storage period. In another example, when the food management device 100 is configured to collect only spectral images regarding ripened food 50a, the server device 200 may perform control regarding ripening state detection and storage environment adjustment for the ripened food 50a while controlling the food management device 100. In this regard, the server device 200 may receive information related to the ripened food 50a, such as at least one spectral image taken of the ripened food 50a and information on the storage environment of the ripened food 50a, from the food management device 100, generate control information for detecting the ripening state of the ripened food 50a and changing the storage environment of the ripened food 50a based on the received information, and transmit the generated information to the storage warehouse 300 directly or through the food management device 100. Meanwhile, if the food management device 100 is designed to directly perform state analysis and storage environment control of the ripened food 50a, the server device 200 may be omitted from the system environment 10.

As described above, the system environment 10 supporting the food management function according to the second embodiment of the present invention can obtain spectral images related to the ripened food 50a by placing the food management device 100 configured to obtain the spectral images of the ripened food 50a in the storage warehouse 300 where the ripened food 50a is stored. In the system environment 10, the food management device 100 can detect the ripening state of the ripened food 50a by perform analysis on the obtained spectral images, thereby enabling the ripening state to be easily confirmed and preventing the marketability of food from deteriorating or food from being discarded due to ripening failure. The system environment 10 of the present invention can support even a manager who has no experience or is not skilled in food ripening to manage the ripening state of the food. In addition, the system environment 10 of the present invention can support easy detection of the food ripening state without the manager having to directly visually check or taste the ripened food 50a or use a separate measuring device.

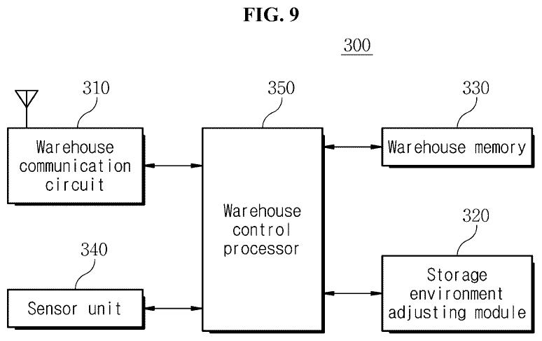

FIG. 9 is a diagram showing an example of components of a storage warehouse capable of storing food according to the second embodiment of the present invention.

Referring to FIG. 9, the storage warehouse 300 includes a warehouse building that forms a certain space in which the ripened food 50a can be stored. In order to detect the ripening state of the ripened food 50a and control storage when the ripened food 50a is stored in the warehouse building, the storage warehouse 300 may include a warehouse communication circuit 310, a storage environment adjusting module 320, a warehouse memory 330, a sensor unit 340, and a warehouse control processor 350.

The warehouse communication circuit 310 may form a communication channel of the storage warehouse 300. For example, the warehouse communication circuit 310 may form a communication channel with at least one of the food management device 100 and the server device 200 and transmit operation information related to the operation of the storage warehouse 300 to the food management device 100 or the server device 200. The operation information of the storage warehouse 300 may include, for example, at least some of the type of ripened food 50a being stored, information on the storage environment for each ripened food 50a, the storage date of the ripened food 50a, and the expected shipping date of the ripened food 50a. The warehouse communication circuit 310 may receive storage environment change information (e.g., setting information for changing the states of the storage environment) related to the ripened food 50a from the food management device 100 or the server device 200.

The storage environment adjusting module 320 may include a module that adjusts the environment of at least some areas of the storage warehouse 300 or an area where the ripened food 50a is stored. For example, the storage environment adjusting module 320 may include at least one of a temperature adjusting device capable of adjusting the temperature of a certain area where the ripened food 50a is placed, a humidity adjusting device capable of adjusting humidity, and a light irradiation device capable of adjusting the amount of light irradiation. The storage environment adjusting module 320 may adjust at least one of temperature, humidity, and light to maintain the freshness of the ripened food 50a for a long time under the control of the warehouse control processor 350.

The warehouse memory 330 may store various data and programs required for the operation of the storage warehouse 300. For example, the warehouse memory 330 may include a program for operating and controlling the at least one adjusting device included in the storage environment adjusting module 320. In addition, the warehouse memory 330 can store at least one program for operating and controlling the sensor unit 340 arranged in the storage warehouse 300. The warehouse memory 330 may store information on the type of the ripened food 50a that is received in the storage warehouse 300, the received date of the ripened food 50a, the location information in the storage warehouse 300 where the ripened food 50a is placed, the number of days of storage of the ripened food 50a, the expected shipping date of the ripened food 50a, etc. In addition, the warehouse memory 330 may store the current storage environment setting information of the ripened food 50a.

The sensor unit 340 may be placed in at least some areas of the storage warehouse 300 and collect various kinds of sensor information related to the ripened food 50a. For example, the sensor unit 340 may include at least one of a location detection sensor capable of identifying a location where the ripened food 50a is placed, and a sensor (e.g., at least one of a temperature sensor, a humidity sensor, and a light detection sensor) capable of sensing a storage environment where the ripened food 50a is placed. The sensor unit 340 may collect sensing information on an area where the ripened food 50a is placed under the control of the warehouse control processor 350, and transmit the collected sensing information to the warehouse control processor 350.

The warehouse control processor 350 may perform various operation controls required for operating the storage warehouse 300. For example, the warehouse control processor 350 may adjust at least some of the temperature, humidity, and light amount of a specific area of the storage warehouse 300 according to the storage warehouse setting information. In this operation, the warehouse control processor 350 may control the sensor unit 340 to sense the temperature, humidity, and light amount of a specific area where the ripened food 50a is placed, and adjust the temperature, humidity, and light amount to correspond to the values set in the setting information. In an example, when the ripened food 50a is received and the storage warehouse setting information is determined, the warehouse control processor 350 may transmit the type of the ripened food 50a, the location information where the food 50 is placed, the expected shipping date of the ripened food 50a, and the current storage environment setting information of the area where the ripened food 50a is placed to the food management device 100 or the server device 200. Upon receiving storage environment change information from the food management device 100 or the server device 200, the warehouse control processor 350 may adjust at least some of the temperature, humidity, and light amount of the storage environment in response to the received storage environment change information. When the ripened food 50a is shipped out, the warehouse control processor 350 may transmit information on the shipping of the ripened food 50a to the food management device 100 or the server device 200, and control the sensor unit 340 and the storage environment control module 320 at the area where the ripened food 50a is placed to be turned off.

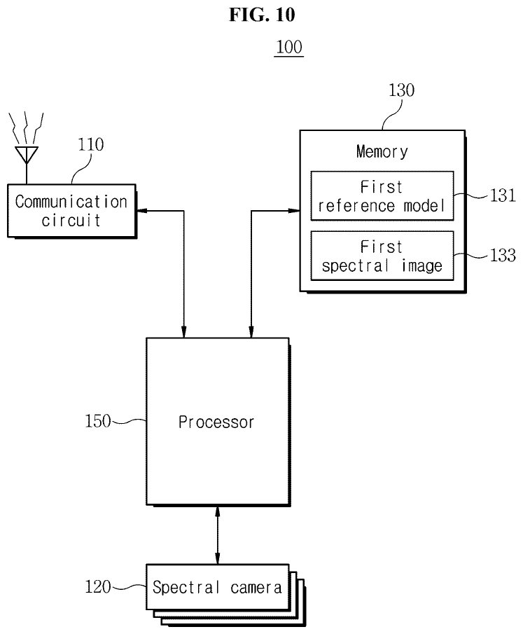

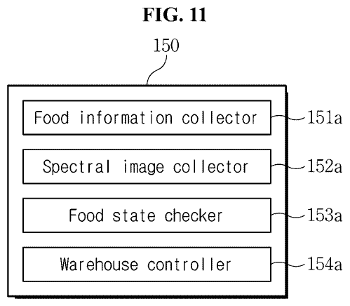

FIG. 10 is a diagram showing an example of components of a food management device according to the second embodiment of the present invention, and FIG. 11 is a diagram showing an example of components of a processor among the components of the food management device according to the second embodiment of the present invention.

First, referring to FIG. 10, the food management device 100 according to the second embodiment of the present invention may include a communication circuit 110, at least one spectral camera 120, a memory 130, and a processor 150. Additionally, the food management device 100 may further include a mounting structure for mounting the at least one spectral camera 120 so that the at least one spectral camera 120 can photograph an area where at least one ripened food 50a is placed. In addition, the food management device 100 may further include a power supply (e.g., a permanent power supply or a battery) required for the operation of at least one of the above-mentioned components, for example, the communication circuit 110, the at least one spectral camera 120, the memory 130, and the processor 150.

The communication circuit 110 can establish a communication channel with the storage warehouse 300 or the server device 200. If the server device 200 is designed to perform the calculation required for the food management function according to the second embodiment of the present invention, the communication circuit 110 may transmit at least one spectral image collected by the at least one spectral camera 120 to the server device 200. On the other hand, the food management function may be performed independently by the food management device 100. In this case, the communication circuit 110 may transmit a message including ripening-related information (e.g., at least one of ripening completion or not, ripening degree, remaining period until target ripening, and ripening environment adjustment) generated during the operation of the food management function to a manager terminal of the food management device 100 or a designated user terminal device in response to the control of the processor 150. Alternatively, the communication circuit 110 may output (or transmit) the message to the server device 200 in response to the control of the processor 150. If the food management device 100 includes a separate output device (e.g., a display or an audio device), the message may be output through the output device.

The communication circuit 110 can receive food-related information of the ripened food 50a from the storage warehouse 300 (e.g., at least one of information on the type of the ripened food 50a received in the storage warehouse 300, the received date of the ripened food 50a, location information in the storage warehouse 300 where the ripened food 50a is placed, the number of days of storage of the ripened food 50a, and the expected shipping date of the ripened food 50a). The communication circuit 110 may transmit change information for changing the storage environment of the ripened food 50a to the storage warehouse 300 in response to the control of the processor 150.

The at least one spectral camera 120 may be arranged to capture a spectral image of at least one ripened food 50a. The at least one spectral camera 120 may include a plurality of spectral cameras, and the plurality of spectral cameras 120 may be arranged in a plurality of areas of the storage warehouse 300 to capture spectral images of a plurality of ripened foods 50a located in the storage warehouse 300, respectively. In an example, when the ripened food 50a is stored at a certain location (e.g., a food rack where the ripened food 50a can be arranged) in the storage warehouse 300, the spectral camera 120 arranged at that location may be activated in response to the control of the processor 150 and then collect spectral images of the ripened food 50a.

The memory 130 can store at least one program or data required for the operation of the food management device 100. For example, the memory 130 may store a control program required for operating the at least one spectral camera 120, and a first spectral image 133 acquired through the at least one spectral camera 120. For example, the memory 130 may store a first reference model 131 corresponding to the target ripening degree of the ripened food 50a. The first reference model 131 may vary depending on the type and target ripening degree of the ripened food 50a. Therefore, when a plurality of types of ripened food 50a are stored in the storage warehouse 300, the first reference model 131 may include models corresponding to the number of the plurality of ripened foods 50a. In addition, even for the same type, the target ripening degree of the ripened food 50a may be different. Therefore, the first reference model 131 may include models for respective target ripening degrees of the ripened food 50a.

The processor 150 may perform at least one of the transmission and processing of signals required for the operation of the food management device 100 and the storage and output of processing results. For example, the processor 150 may control the acquisition of spectral images related to the ripened food 50a using the at least one spectral camera 120 in response to at least one of guidance information from the storage warehouse 300 (e.g., information notifying a situation in which a new ripened food 50a has arrived) or a request from the server device 200. The processor 150 may detect the state of the ripened food 50a based on the acquired at least one spectral image, determine whether it corresponds to a predefined ripening degree, and output a corresponding notification. In this regard, the processor 150 may include components as illustrated in FIG. 11.

Referring to FIG. 11, the processor 150 may include at least one of a food information collector 151a, a spectral image collector 152a, a food state checker 153a, and a warehouse controller 154a.

The food information collector 151a may collect information on the ripened food 50a from the storage warehouse 300. In this regard, the food information collector 151a may maintain a communication channel with the warehouse communication circuit 310 of the storage warehouse 300. Upon receiving ripened food-related information from the storage warehouse 300, the food information collector may store the received information in the memory 130 and request the spectral image collector 152a to collect spectral images. In this process, the food information collector 151a may extract location information on where the food is stored from the ripened food-related information provided by the storage warehouse 300 and transmit the extracted location information to the spectral image collector 152a.

The spectral image collector 152a can control acquisition of a spectral image using the at least one spectral camera 120. For example, when the spectral image collector 152a receives location information of a newly received ripened food 50a from the food information collector 151a, it may process acquisition of a spectral image for the newly received ripened food 50a. In this process, the spectral image collector 152a may control the spectral camera 120 placed at a location of the ripened food 50a to acquire a spectral image for the ripened food 50a. The spectral image collector 152a may provide the acquired spectral image to the food state checker 153a. The spectral image collector 152a may collect spectral images at a time requested by the food state checker 153a or according to a predefined period of time, and notify the spectral image collection to the food state checker 153a. In this operation, the spectral image collector 152a may store the collected spectral images as the first spectral image 133 in the memory 130. When the ripened food 50a of a certain location is shipped out, the spectral image collector 152a may control the spectral camera 120 placed to photograph the location where the ripened food 50a is placed to be deactivated.

Upon receiving a notification of the spectral image collection from the spectral image collector 152a, the food state checker 153a can perform ripening state detection for the ripened food 50a based on the analysis on the first spectral image 133 stored in the memory 130. For example, the food state checker 153a may compare the first spectral image 133 with the first reference model 131 previously stored in the memory 130. For example, the food state checker 153a may compare the spectrum of the first spectral image 133 with the spectrum of the first reference model 131 to determine whether it is within a specified similarity range. The first reference model 131 may include, for example, a model corresponding to the ripening degree of a specific ripened food 50a. For example, a device that provides the first reference model 131 may collect a plurality of spectral images for meat aged for a certain period of time (e.g., 30 days, 90 days, 120 days, etc.) under specified conditions (e.g., low-temperature aging conditions), perform clustering modeling through an artificial neural network on spectra of the collected spectral images, and generate the first reference model 131 based on the average value (e.g., centroid) and standard deviation of clustering models. The first reference model 131 may be received by the food management device 100 from, for example, an external server device that provides the reference model, or may be directly generated by the food management device 100 through confirmation by a professional human resource and the period and conditions for the ripened food 50a stored in the storage warehouse 300. When a new first spectral image 133 is acquired, the food state checker 153a may determine whether the current state of the ripened food 50a is the targeted optimal state (or ripening complete state) through machine learning using the acquired first spectral image 133. For example, the food state checker 153a may perform spectrum analysis on the first spectral image 133 to determine the similarity with the first reference model 131. In relation to the detection of the current ripening state of the ripened food 50a, the food state checker 153a may add information on the storage environment in which the ripened food 50a is stored. The storage environment information may include at least one of temperature, humidity, and illuminance (or light quantity).

The food state checker 153a may calculate the remaining period until the target ripening completion time based on the current ripening state of the ripened food 50a. In this regard, the first reference model 131 may include models for each period of the ripened food 50a. For example, the food state checker 153a may calculate the remaining period by checking a period difference between the period-specific model matching the currently detected first spectral image 133 and the target model. Additionally or alternatively, a change in the storage environment for each ripening period of the ripened food 50a may be required. Therefore, the food state checker 153a may check whether a change in the storage environment is required according to the current ripening state of the ripened food 50a. In this regard, the memory 130 may store information on the change in the storage environment for each ripening period. If the ripening period of the detected ripened food 50a changes, the food state checker 153a may check the storage environment corresponding to the changed ripening period from information on the storage environment for each ripening period previously stored in the memory 130, and if a change is necessary, generate change information and transmit it to the warehouse controller 154a.

In addition, the food state checker 153a may notify the result of the analysis on the first spectral image 133 to a designated user. For example, the food state checker 153a may provide a message including at least one of information on the current ripening degree of the ripened food 50a, the target ripening degree of the ripened food 50a, the remaining time until the current target ripening, the expected shipping date of the ripened food 50a, and the current storage environment of the ripened food 50a to a designated user (e.g., a manager terminal of the storage warehouse 300, a manager terminal of the food management device 100, etc.). Alternatively, the food state checker 153a of the food management device 100 including an output device (e.g., a display) may provide the message to the output device.

The warehouse controller 154a may receive change information for changing the storage environment from the food state checker 153a. The warehouse controller 154a may create a control message including the received change information and transmit the created control message to the storage warehouse 300. The control message may include, for example, information requesting to adjust at least some of the temperature, humidity, and light amount of an area of the storage warehouse 300 where a certain ripened food 50a is placed.

As described above, the food management device 100 according to the second embodiment of the present invention can collect and analyze the spectral images of the ripened food 50a stored in at least one place of the storage warehouse 300 to check whether the state of the ripened food 50a corresponds to a specified state, and provide a related message or control the storage warehouse 300. Through this, the food management device 100 can support even a worker without prior knowledge or experience in the ripened food 50a to manage the ripening degree of the ripened food 50a, and can support stable ripening of the ripened food 50a by allowing a worker without expert knowledge to check whether ripening of the ripened food 50a is complete.

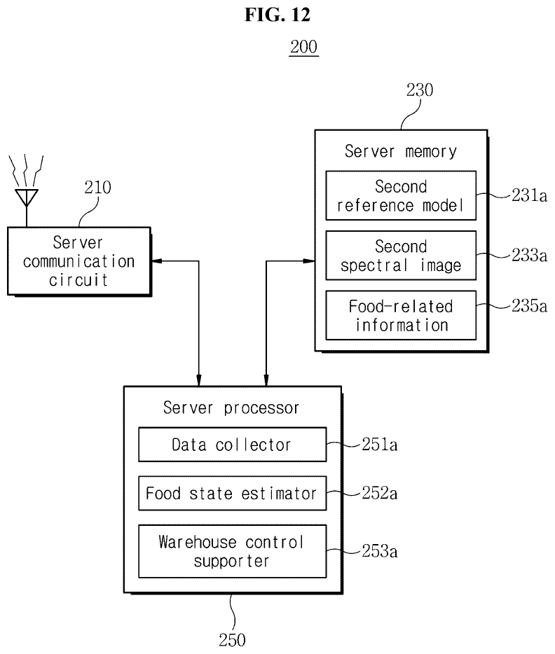

Meanwhile, in the above description, the food management device 100 is described as detecting the state of the ripened food 50a stored in the storage warehouse 300 to inform whether the ripening of the ripened food 50a is complete and the degree of ripening, and directly controlling the change of the storage environment as needed, but the present invention is not limited thereto. For example, the food management device 100 may be designed to control only the spectral camera 120 to collect spectral images of the ripened food 50a and transmit the collected spectral images to the server device 200. In this case, the server device 200 may support the state detection and management function for the ripened food 50a based on the components as illustrated in FIG. 12.

FIG. 12 is a diagram showing an example of components of a server device according to the second embodiment of the present invention. As described above, if the food management device 100 is designed to perform the management function for the ripened food 50a according to the second embodiment of the present invention, the server device 200 may be omitted.

Referring to FIG. 12, the server device 200 of the present invention may include a server communication circuit 210, a server memory 230, and a server processor 250.

The server communication circuit 210 may directly form a communication channel with the food management device 100. Additionally, the server communication circuit 210 may form a communication channel with the storage warehouse 300 or communicate with the storage warehouse 300 via the food management device 100. The server communication circuit 210 may receive at least one spectral image from the food management device 100 in response to a specified period or occurrence of a predefined event. Additionally, the server communication circuit 210 may receive at least one food-related information from the storage warehouse 300. The server communication circuit 210 may transmit storage environment change information to the storage warehouse 300 in response to control of the server processor 250.

The server memory 230 can store at least one program or data required for the operation of the server device 200. For example, the server memory 230 may store at least one of a second spectral image 233a collected and transmitted by the food management device 100, a second reference model 231a for comparison with the second spectral image 233a, and food-related information 235a stored in the storage warehouse 300. The second spectral image 233a may be an image corresponding to the first spectral image 133 described above in the food management device 100. For example, the second spectral image 233a may include a spectral image currently acquired for checking the ripening state of the ripened food 50a. The second reference model 231a may correspond to the first reference model 131 of the food management device 100 described above. The food-related information 235a is information about the ripened food 50a provided by the storage warehouse 300, and may include at least some of the type of the ripened food 50a, the received date of the ripened food 50a, the expected shipping date of the ripened food 50a, the target ripening degree of the ripened food 50a, and the storage environment information (e.g., at least one of temperature, humidity, and light quantity) for the ripened food 50a.

The server processor 250 can control the transmission and processing of signals required for the operation of the server device 200, storage or transmission of results, or transmission of messages corresponding to results. In this regard, the server processor 250 may include a data collector 251a, a food state detector 252a, and a warehouse control supporter 253a.

The data collector 251a can receive food-related information from the storage warehouse 300. In this regard, the data collector 251a may form a communication channel with the storage warehouse 300, and when a new ripened food 50a is received in the storage warehouse 300 or when the ripened food 50a being stored is shipped out, the data collector 251a may receive food-related information from the storage warehouse 300. The data collector 251a may temporarily or semi-permanently store the received food-related information 235a in the server memory 230. Upon receiving the food-related information from the storage warehouse 300, the data collector 251a may request a spectral image corresponding to the food from the food management device 100. The data collector 251a may request and receive a spectral image from the food management device 100 according to the occurrence of a designated event (e.g., occurrence of an event requesting to check the ripening state of the ripened food 50a) after the ripened food 50a is placed in the storage warehouse 300. Alternatively, the data collector 251a may receive a spectral image of the ripened food 50a from the food management device 100 at regular time intervals. Upon receiving a spectral image, the data collector 251a may store it as the second spectral image 233a in the server memory 230.

When the food state detector 252a is notified of the collection of the second spectral image 233a from the data collector 251a, it may perform an analysis on the second spectral image 233a stored in the server memory 230. For example, the food state detector 252a may compare the spectrum of the second spectral image 233a with the second reference model 231a corresponding to the target ripening degree of the corresponding ripened food 50a to determine whether the ripening of the ripened food 50a stored in the storage warehouse 300 is complete or to determine the ripening degree. In this regard, the second reference model 231a may include a completion model corresponding to the ripening completion time of the ripened food 50a or models representing the progress of the ripening time of the ripened food 50a. The second reference model 231a may be generated by the food management device 100 or received from a separate external server device that provides the reference model. Alternatively, the server device 200 may accumulate and store spectral images of the ripened foods 50a by period and generate the reference model based on the accumulated spectral images. In this process, the server device 200 may also store storage environment information to secure a reference model of how ripening of the ripened food 50a progresses in which environment.

The food state detector 252a may provide information based on the comparison result between the second spectral image 233a and the second reference model 231a to a designated terminal device. For example, the food state detector 252a may provide at least one of whether the ripening of the ripened food 50a is complete, the current ripening degree of the ripened food 50a, and the estimated remaining period until the target ripening of the ripened food 50a to a terminal device of a manager of the storage warehouse 300, a terminal device of a manager of the food management device 100, a terminal device of a manager who requests to check the ripening information of the ripened food 50a, or an output device (e.g., a display device) connected to the server device 200. If the ripened food 50a is a food that requires a change in storage environment according to the ripening period, the food state detector 252a may generate change information for changing the storage environment and transmit it to the warehouse control supporter 253a. In this regard, the server memory 230 may store and manage information on the storage environment according to the ripening degree of the ripened food 50a.

When the warehouse control supporter 253a receives change information for changing the storage environment of the ripened food 50a from the food state detector 252a, the warehouse control supporter 253a may transmit, to the storage warehouse 300, storage environment change information requesting a change in the storage environment based on the change information. In this process, the warehouse control supporter 253a may check the food-related information 235a to identify environmental factors (e.g., at least one of temperature, humidity, and light intensity) that need to be changed based on the current storage environment of the ripened food 50a, create the storage environment change information requesting a change in at least one of the storage environment factors, and transmit the created information to the storage warehouse 300.

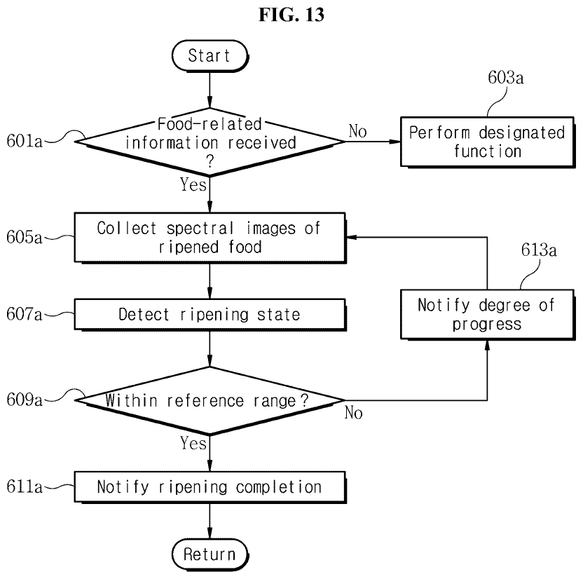

FIG. 13 is a diagram showing an example of a food management method of a food management device according to the second embodiment of the present invention.

Referring to FIG. 13, in a method of operating the food management device 100 related to the food management method according to the second embodiment of the present invention, the processor 150 of the food management device 100 may check in step 601a whether food-related information is received. In this regard, the food management device 100 may establish a communication channel with the storage warehouse 300, and when a new ripened food 50a is received in the storage warehouse 300, the storage warehouse 300 may transmit the food-related information to the food management device 100. Here, the food-related information may include at least some of the name of the ripened food 50a, the received date of the ripened food 50a, the placed location of the ripened food 50a, and the expected shipping date of the ripened food 50a. If the food-related information is not received, the processor 150 of the food management device 100 may perform a designated function in step 603a. For example, the food management device 100 may perform a management function on previously registered ripened food 50a and check whether the ripened food 50a being managed has reached the target degree of ripening. Alternatively, the food management device 100 may receive a reference model on newly received ripened food 50a from an external server device and store/manage it.

Upon receiving the food-related information from the storage warehouse 300, the processor 150 of the food management device 100 may collect spectral images of the ripened food 50a in step 605a. In this process, the food management device 100 may check the food-related information to identify the location of the ripened food 50a, identify the spectral camera 120 disposed to be able to photograph the corresponding location, and activate the spectral camera 120 at that location. The processor 150 may control the activated spectral camera 120 to collect spectral images at a certain event occurrence time or at a predefined regular cycle from the time the ripened food 50a is received. In relation to this function, the food management device 100 may store and manage location information about a plurality of spectral cameras disposed to photograph a plurality of locations where the ripened food 50a can be placed in the storage warehouse 300, and about an area that each of the spectral cameras can photograph.

In step 607a, the processor 150 of the food management device 100 may perform an analysis on the collected spectral images to detect the ripening state of the ripened food 50a. For example, the processor 150 may compare the spectral image spectrum of the ripened food 50a with reference models pre-stored in the memory 130 to determine which reference model corresponds to the spectral image spectrum. The reference models may include models generated through clustering modeling of at least one spectral image acquired at each time according to the ripening degree of the ripened food 50a. The processor 150 may detect the ripening state of the ripened food by referring to the reference model having the most similar cluster.

In step 609a, the processor 150 may check whether the detected ripening state value is within a target reference range. If the detected ripening state value is within the target reference range, the processor 150 may return to step 611a and perform notification according to the ripening completion. For example, the processor 150 may transmit a ripening completion message for the ripened food 50a to a pre-designated manager terminal device. After the notification of the ripening completion, if the ripened food 50a is shipped out from the storage warehouse 300, the food management function for the ripened food 50a may be terminated. If the ripened food 50a is not shipped out, the processor 150 may repeatedly perform operations after step 605a until the ripened food 50a is shipped out.

If the ripening status value is out of the reference range in step 609a, the processor 150 of the food management device 100 may provide notification on the degree of ripening in step 613a. Here, if the ripening degree is less than the reference range, the processor 150 may create a message about an underripe state and transmit it to a pre-designated manager terminal device (e.g., at least one of a terminal device of a manager of the storage warehouse 300, a terminal device of a manager of the food management device 100, or a terminal device of a designated manager who must confirm the report on ripening). Additionally or alternatively, the processor 150 may compare the currently identified ripening time with the target ripening time, calculate the remaining time until the ripening target time, and transmit the calculated remaining time information to the designated manager terminal device. Further, when the storage environment needs to be changed according to a change in the ripening stage of the ripened food 50a, the processor 150 may generate change information for changing at least some factors of the storage environment and provide the change information to the storage warehouse 300.

Meanwhile, if the ripening state value exceeds the reference range, i.e., in case of overripe, the processor 150 of the food management device 100 may notify the overripe state of the ripened food 50a to a manager terminal that should receive ae report on the overripe state of the ripened food 50a. In this process, the processor 150 may provide a message suggesting the movement or disposal of the ripened food 50a according to the overripeness to the designated manager terminal device. Alternatively, the processor 150 may provide change information requesting a change in the storage environment of the overripe food to the designated manager terminal device or the storage warehouse 300. The above-described operation may be repeatedly performed until the ripened food 50a is shipped out from the storage warehouse 300.

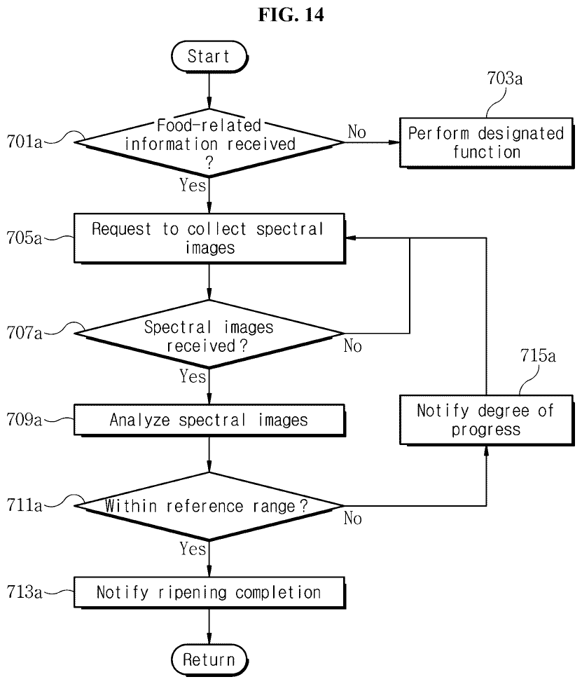

FIG. 14 is a diagram showing an example of server device operation related to the food management method according to the second embodiment of the present invention.

Referring to FIG. 14, in step 701a, the server processor 250 of the server device 200 may check whether the food-related information is received. In this regard, the server device 200 may maintain a communication channel with the storage warehouse 300. If the food-related information is not received, the server processor 250 may perform a designated function in step 703a. For example, the server processor 250 may perform ripening state management for previously registered ripened food 50a.

When the food-related information is received, the server processor 250 may request in step 705a the food management device 100 to collect spectral images. In this regard, the server device 200 may establish a communication channel with the food management device 100 and request the food management device 100 to collect spectral images corresponding to the received food-related information. In this operation, the server device 200 may provide the food management device 100 with location information of the ripened food 50a in the storage warehouse 300. The food management device 100 may identify a spectral camera mapped to the location information of the ripened food 50a in the storage warehouse 300, activate the corresponding spectral camera to acquire a spectral image of the newly received ripened food 50a, and transmit the acquired spectral image to the server device 200.

In step 707a, the server processor 250 may check whether a spectral image is received, and if no spectral image is received within a specified time, the server processor 250 may re-perform step 705a. Meanwhile, the server processor 250 may request the collection of spectral images more than a predefined number of times, and if no spectral image is received in response, determine that a failure has occurred in the food management device 100. Then, the server processor 250 may take related measures (e.g., notify the failure to a management terminal that manages the food management device 100).

When the spectral image is received, the server processor 250 may perform spectral image analysis in step 709a. In this regard, the server processor 250 may compare the currently collected spectral image with the reference model for the ripened food 50a.

In step 711a, the server processor 250 may check whether the ripening state value of the detected ripened food 50a is within a target reference range through spectrum comparison analysis. If the spectrum comparison analysis result is within the reference range, the server processor 250 may return to step 713a and notify the completion of ripening to a designated manager terminal device. Thereafter, if the server processor 250 receives a shipping message for the ripened food 50a with ripening completed from the storage warehouse 300, the server processor 250 may terminate the food management function for the ripened food 50a. If the shipping message is not received, the server processor 250 may re-perform the operation after step 705a.

If the spectrum comparison analysis result shows that the ripening state value is out of the reference range, the server processor 250 may notify the progress according to whether it is below or above the reference range in step 715a. For example, if the ripening state is below the reference range, the server processor 250 may transmit a message including at least one of a message indicating an underripe state, the remaining time until ripening completion, current storage environment setting information, and change information for changing the storage environment according to the underripe period to a designated manager terminal device. If the ripening state exceeds the reference range, the server processor 250 may provide a message including at least one of a message indicating an overripe state and storage environment change information to the manager terminal device or the storage warehouse 300.

As described above, the food management method according to the second embodiment of the present invention can determine the optimal state of the food (or the optimal ripening state, the target ripening state) based on the spectral images taken of the ripened food. In addition, the food management method of the present invention can estimate the timing of the optimal ripening state or the target ripening state based on information about the current ripening state of the ripened food.

While the description contains many specific implementation details, these should not be construed as limitations on the scope of the present disclosure or of what may be claimed, but rather as descriptions of features that may be specific to particular embodiments of particular disclosure.

Also, although the description describes that operations are performed in a predetermined order with reference to a drawing, it should not be construed that the operations are required to be performed sequentially or in the predetermined order, which is illustrated to obtain a preferable result, or that all of the illustrated operations are required to be performed. In some cases, multi-tasking and parallel processing may be advantageous. Also, it should not be construed that the division of various system components are required in all types of implementation. It should be understood that the described program components and systems are generally integrated as a single software product or packaged into a multiple-software product.

The description shows the best mode of the present disclosure and provides examples to illustrate the present disclosure and to enable a person skilled in the art to make and use the present disclosure. The present disclosure is not limited by the specific terms used herein. Based on the above-described embodiments, one of ordinary skill in the art can modify, alter, or change the embodiments without departing from the scope of the present disclosure.

Accordingly, the scope of the present disclosure should not be limited by the described embodiments and should be defined by the appended claims.

### REFERENCE NUMERALS

10: System environment
50: Food
50a: Ripened food
100: Food management device
110: Communication circuit
120: Spectral camera
130: Memory
140: Sensor unit
150: Processor
200: Server device
210: Server communication circuit
230: Server memory
250: Server processor
300: Storage warehouse
310: Warehouse communication circuit
320: Warehouse memory
330: Storage environment adjusting module
340: Sensor unit

What is claimed is:

1. A food management device supporting food state tracking based on a spectral image, the device comprising:
at least one spectral camera for acquiring spectral images of food received in a storage warehouse; and
a processor functionally connected to the at least one spectral camera,
the processor configured to:
receive food-related information of the received food from the storage warehouse,
acquire the spectral images of the food at regular intervals by activating the at least one spectral camera, and
monitor a change in a state of the food by analyzing the acquired spectral images.

2. The device of claim 1, wherein the processor is configured to:
identify a location of the received food from the food-related information, and
activate the spectral camera disposed at the location of the food.

3. The device of claim 2, wherein the processor is configured to:
deactivate the spectral camera disposed at the location when receiving a message regarding shipment of the food from the storage warehouse.

4. The device of claim 1, wherein the processor is configured to:
acquire an initial spectral image at a time the food is received, and
track the change in the state of the food by comparing the initial spectral image with the spectral images acquired at regular intervals thereafter.

5. The device of claim 1, wherein the processor is configured to:
if the change in the state of the food is greater than or equal to a predefined reference value, notify the change in the state of the food to a designated terminal.

6. The device of claim 1, wherein the processor is configured to:
if the change in the state of the food is greater than or equal to a predefined reference value, collect information on a storage environment corresponding to the changed state of the food, create change information for changing a storage environment of the food based on the collected information, and transmit the change information to the storage warehouse.

7. The device of claim 6, wherein the processor is configured to:
create the change information for changing at least one of temperature, humidity, and light amount in the storage environment of the food.

8. A food state tracking method based on a spectral image, the method comprising:
by a processor of a food management device that manages storage of food,
establishing a communication channel with a storage warehouse where the food is stored;
receiving food-related information on the food received in the storage warehouse from the storage warehouse;
acquiring spectral images of the food at regular intervals by using a spectral camera disposed to capture the spectral images of the food; and
monitoring a change in a state of the food by analyzing the acquired spectral images.

9. The method of claim 8, wherein acquiring spectral images includes:
identifying a location of the received food from the food-related information; and
activating the spectral camera disposed at the location of the food.

10. The method of claim 9, further comprising:
receiving a message regarding shipment of the food from the storage warehouse; and
deactivating the spectral camera disposed at the location.

11. The method of claim 8, wherein monitoring a change in a state of the food includes at least one of:
if the change in the state of the food is greater than or equal to a predefined reference value, notifying the change in the state of the food to a designated terminal; and
if the change in the state of the food is greater than or equal to a predefined reference value, collecting information on a storage environment corresponding to the changed state of the food, creating change information for changing a storage environment of the food based on the collected information, and transmitting the change information to the storage warehouse.

12. A food management device supporting a food management function based on a spectral image, the device comprising:
at least one spectral camera for acquiring spectral images of a ripened food received in a storage warehouse; and
a processor functionally connected to the at least one spectral camera,
the processor configured to:
when the ripened food is received at a point of the storage warehouse,
activate a spectral camera disposed to photograph the point of the storage warehouse,
acquire a spectral image of the ripened food through the activated spectral camera,
detect a ripening state of the ripened food by comparing the spectral image with a pre-stored reference model, and
output a message according to the ripening state.

13. The device of claim 12, wherein the processor is configured to:
check a current degree of ripening through comparison between the detected ripening state and the reference model, and
if the degree of ripening is within a target reference range, output a message based on completion of ripening.

14. The device of claim 13, wherein the processor is configured to:
if the degree of ripening is below the reference range, output a message including at least one of a message indicating an underripe state and a remaining time until a time of the ripening completion.

15. The device of claim 14, wherein the processor is configured to:
if a storage environment of the storage warehouse needs to be changed based on the underripe state, create change information related to the change of the storage environment and transmit the change information to the storage warehouse.

16. The device of claim 13, wherein the processor is configured to:
if the degree of ripening exceeds the reference range, output a message indicating an overripe state.

17. A food management method based on a spectral image, the method comprising:
by a processor of a food management device,
receiving food-related information related to storage of a ripened food from a storage warehouse;
activating a spectral camera disposed to photograph a point of the storage warehouse, based on the food-related information;
acquiring a spectral image of the ripened food through the activated spectral camera;
detecting a ripening state of the ripened food by comparing the spectral image with a pre-stored reference model; and
outputting a message according to the ripening state.

18. The method of claim 17, wherein detecting a ripening state of the ripened food includes:
checking a current degree of ripening through comparison between the detected ripening state and the reference model; and
if the degree of ripening is within a target reference range, outputting a message based on completion of ripening, if the degree of ripening is below the reference range, outputting a message including at least one of a message indicating an underripe state and a remaining time until a time of the ripening completion, or if the degree of ripening exceeds the reference range, outputting a message indicating an overripe state.

19. The method of claim 18, further comprising:
if a storage environment of the storage warehouse needs to be changed based on the underripe state, creating change information related to the change of the storage environment and transmitting the change information to the storage warehouse.

20. A food management device supporting a food management function based on a spectral image, the device comprising:
a server communication circuit establishing a communication channel with a storage warehouse and a food management device; and
a server processor functionally connected to the server communication circuit,
the server processor configured to:
when receiving a message about reception of a ripened food from the storage warehouse, request a spectral image of the ripened food from the food management device that controls the spectral camera disposed at a location where the ripened food is received,
when receiving the spectral image from the food management device, detect a ripening state of the ripened food by comparing the spectral image with a pre-stored reference model, and
output a message based on the detected ripening state.

21. The device of claim 20, wherein the server processor is configured to:
check a current degree of ripening through comparison between the detected ripening state and the reference model, and
if the degree of ripening is within a target reference range, output a message based on completion of ripening,
if the degree of ripening is below the reference range, output a message including at least one of a message indicating an underripe state and a remaining time until a time of the ripening completion, or
if the degree of ripening exceeds the reference range, output a message indicating an overripe state.
